(19) United States
(12) Patent Application Publication
(10) Pub. No.: US 2013/0317581 A1
(43) Pub. Date: Nov. 28, 2013

(54) METHODS AND DEVICES RELATING TO NON-INVASIVE ELECTRICAL NERVE STIMULATION

(71) Applicant: Andrew Redington, Toronto (CA)

(72) Inventor: Andrew Redington, Toronto (CA)

(73) Assignee: The Hospital for Sick Children, Toronto, ON (CA)

(21) Appl. No.: 13/997,743

(22) PCT Filed: Dec. 22, 2011
(86) PCT No.: PCT/IB2011/003280

§ 371 Date: Jun. 25, 2013

Related U.S. Application Data
(60) Provisional application No. 61/428,469, filed on Dec. 30, 2010.

(51) Int. Cl. A61N 1/04

(52) U.S. Cl. CPC A61N 1/0456
USPC 607/115

(57) ABSTRACT

The invention provides, in certain aspects, methods and devices relating to the use of non-invasive electrical nerve stimulation in subjects to reduce ischemic and/or reperfusion injury and restenosis, and to improve physical performance.

### RELATED APPLICATIONS

This application claims the benefit of U.S. Provisional Application Ser. No. 61/428,469, filed on Dec. 30, 2010, entitled “METHODS AND DEVICES RELATING TO NON-INVASIVE ELECTRICAL NERVE STIMULATION”, the entire contents of which are incorporated by reference herein.

### BACKGROUND OF INVENTION

Ischemic diseases are significant causes of mortality in industrialized nations. It is well established that tissue damage results from ischemia (i.e., stoppage or severe reduction of blood flow to the tissue) followed by reperfusion (i.e., reflow of blood to the tissue). Ischemia and reperfusion causes disturbance of microcirculation with ensuing tissue damage and organ dysfunction. Organs such as the kidney, heart, liver, pancreas, lung, brain and intestine are known to sustain damage following ischemia and reperfusion.

### SUMMARY OF INVENTION

Aspects of the invention provide for the use of non-invasive electrical nerve stimulation, following an ischemic event, to reduce ischemic and/or reperfusion injury associated with or resulting from the ischemic event. An ischemic event is any event that results in a reduction in blood flow to one or more tissues and/or organs of the body. The reduction may be partial or it may be complete (i.e., a stoppage of blood flow to a tissue and/or organ). In important embodiments, the ischemic event is a myocardial infarction. Non-invasive electrical nerve stimulation may be performed once or more than once, on a single day or on multiple days. These aspects of the invention can be used to reduce short and/or long term ischemic and/or reperfusion injury associated with or resulting from the ischemic event.

Thus, in one aspect, the invention provides a method comprising performing non-invasive electrical nerve stimulation on a subject to reduce ischemic and/or reperfusion injury in the subject, wherein the ischemic and/or reperfusion injury is associated with an ischemic event in the subject. The non-invasive electrical nerve stimulation is performed at least once after the ischemic event, and more preferably multiple times after the ischemic event. If performed once after the ischemic event, it is performed more than three hours after the ischemic event. If performed multiple times after the ischemic event, it may be performed at least once within 1, 2, 3, or more hours after the ischemic event. Non-invasive electrical nerve stimulation may be performed one or more times within a period of time that is 3-24 hours, 6-24 hours, 6-12 hours, or 12-24 hours after the ischemic event. Non-invasive electrical nerve stimulation may be performed one or more times following the first 24 hour period following the ischemic event. The non-invasive electrical nerve stimulation may be additionally performed during the ischemic event. The ischemic event may be, but is not limited to, a myocardial infarction, stroke, stenosis, surgery including but not limited to cardiovascular surgery, or other medical intervention that causes ischemia in a tissue and/or organ of a subject. The timing of the non-invasive electrical nerve stimulation will depend upon the nature of the ischemic event, as described in greater detail below.

In one embodiment, the subject is having a myocardial infarction. In one embodiment, the subject has had a myocardial infarction. In one embodiment, the non-invasive electrical nerve stimulation is performed on the subject during and after a myocardial infarction. In one embodiment, the non-invasive electrical nerve stimulation is performed on the subject after a myocardial infarction. The non-invasive electrical nerve stimulation may be performed during the ischemic phase and/or the reperfusion phase of a myocardial infarction. The non-invasive electrical nerve stimulation may be performed later than three hours after the myocardial infarction but within a day, within two days, within three days, within four days, within five days, within six days, or within seven days after the myocardial infarction, whether or not non-invasive electrical nerve stimulation was also performed during the myocardial infarction. The non-invasive electrical nerve stimulation may be repeated any number of times during the hours, days or weeks after the myocardial infarction. In some embodiments, the non-invasive electrical nerve stimulation is performed during the myocardial infarction, and then every day, every two days, or every three days after the myocardial infarction. The non-invasive electrical nerve stimulation may be performed in a predetermined manner (e.g., once every day following a myocardial infarction) or it may be performed more randomly after the myocardial infarction.

In one embodiment, the subject is having a stroke. In one embodiment, the subject has had a stroke. In one embodiment, the non-invasive electrical nerve stimulation is performed on the subject during and after a stroke. In one embodiment, the non-invasive electrical nerve stimulation is performed on the subject after the stroke. The non-invasive electrical nerve stimulation may be performed during the ischemic phase and/or the reperfusion phase of the stroke. The non-invasive electrical nerve stimulation may be performed later than three hours after the stroke but within a day, within two days, within three days, within four days, within five days, within six days, or within seven days after the stroke, whether or not non-invasive electrical nerve stimulation was also performed during the stroke. The non-invasive electrical nerve stimulation may be repeated any number of times during the hours, days or weeks after the stroke. In some embodiments, the non-invasive electrical nerve stimulation is performed during the stroke, and then every day, every two days, or every three days after the stroke. The non-invasive electrical nerve stimulation may be performed in a predetermined manner (e.g., once every day following a stroke) or it may be performed more randomly after the stroke.

In one embodiment, the subject is having a surgery that involves partially or completely blocking blood flow to a tissue and/or organ or a surgery that is associated with an elevated risk of such a blockage during or after the surgery. The non-invasive electrical nerve stimulation may be performed on the subject during and after the surgery, or after the surgery. The non-invasive electrical nerve stimulation may be performed later than three hours after the surgery but within a day, within two days, within three days, within four days, within five days, within six days, or within seven days after the surgery, whether or not non-invasive electrical nerve stimulation was also performed during the surgery. The non-invasive electrical nerve stimulation may be repeated any number of times during the hours, days or weeks after the surgery. In some embodiments, the non-invasive electrical nerve stimulation is performed during the surgery, and then every day, every two days, or every three days after the surgery. The non-invasive electrical nerve stimulation may be performed in a predetermined manner (e.g., once every day following the surgery) or it may be performed more randomly after the surgery. The surgery may be emergency or elective surgery.

In important embodiments, the non-invasive electrical nerve stimulation is performed repeatedly on a subject. In some embodiments, the non-invasive electrical nerve stimulation is performed one or more times daily, one or more times every two days, or one or more times every three days, one or more times every four days, one or more times every five days, one or more times every six days, or one or more times every week. In some embodiments, one or more non-invasive electrical nerve stimulations are performed on a daily basis for one month or every other day for one month. On any given day, one, two, three, four, five, or more non-invasive electrical nerve stimulations may be performed. The non-invasive electrical nerve stimulation may be performed repeatedly more than three hours after the ischemic event.

The non-invasive electrical nerve stimulations may be performed over the course of a month, two months, three months, four months, five months, six months, a year or longer, including for the life time of the subject.

As used herein and as described in greater detail herein, a single non-invasive electrical nerve stimulation is comprised of one or more cycles of a nerve stimulation period followed by a rest period. The number of cycles may be one, two, three, four, five, or more cycles.

The non-invasive electrical nerve stimulation may be performed at virtually any region on the body that is amenable to the non-invasive procedure. In some embodiments, it is performed transcutaneously. In some embodiments, it is performed at a region that is remote from the tissue and/or organ being treated or protected. As an example, if the subject is having a myocardial infarction, then the non-invasive electrical nerve stimulation may be performed on a limb (e.g., arm or leg). It may be performed on scalp, face, ear, torso including chest and back, hand, arm, buttock, leg, or foot. When performed repeatedly, the non-invasive electrical nerve stimulations may be performed at the same or at different regions on the body. In some embodiments, two or more non-invasive electrical nerve stimulations are performed simultaneously at different regions on the body. The non-invasive electrical nerve stimulation may be performed using one device or it may be performed using two or more devices.

The ischemic event may be a myocardial ischemic event, a cerebral ischemic event resulting from, for example stroke, head trauma or carotid stenosis, an intestinal ischemic event, a renal ischemic event, or a tissue ischemic event resulting from, for example, cardiac arrest, sepsis or shock. The invention further contemplates reducing ischemic and/or reperfusion injury in subjects having ischemia-related conditions such as but not limited to Alzheimer's disease, Parkinson's disease, cerebral hemorrhage, hemorrhage infarction, hypertensive hemorrhage, hemorrhage due to rupture of intracranial vascular abnormalities, subarachnoid hemorrhage (e.g., due to rupture of intracranial arterial aneurysms), hypertensive encephalopathy, cardiogenic thromboembolism, spinal stroke and spinal cord injury, diseases of cerebral blood vessels including for example atherosclerosis, vasculitis, macular degeneration, and superaventicular tachyarrhythmia.

A surgery that involves partially or completely blocking blood flow to a tissue and/or organ in a subject includes but is not limited to cardiovascular surgery such as cardiac surgery, coronary artery bypass graft, heart valve surgery, heart transplantation, surgery for congenital heart disease, as well as relatively minimally invasive procedures such as intravascular stent placement, and angioplasty including balloon angioplasty. Other surgeries include lung surgery, liver surgery, kidney surgery, pancreas surgery, bowel surgery, lung transplant, liver transplant, kidney transplant, pancreas transplant.

In preferred embodiments of various aspects of the invention, the subject is a human. The subject may or may not be experiencing angina including refractory angina. The subject may or may not have a history of angina including refractory angina. The subject may or may not be experiencing pain, including muscle pain, further including back pain. The subject may or may not have a history of pain, including muscle pain, further including back pain. The subject may or may not have a pace maker or other implanted device that controls cardiac rhythm and/or that stimulates nerves.

In some embodiments, the subject has undergone and/or is undergoing and/or will undergo deliberate mechanical ischemic conditioning, whether local or remote to the location of nerve stimulation and/or to the location of the tissue and/or organ being treated or protected from ischemic/reperfusion injury. In some embodiments, deliberate mechanical ischemic conditioning is performed in combination with non-invasive electrical nerve stimulation, whether during and after the ischemic event, or after the ischemic event.

Aspects of the invention provide methods for reducing restenosis in a subject comprising performing non-invasive electrical nerve stimulation on a subject having or likely to experience restenosis. Reducing restenosis may comprise reducing the incidence of restenosis compared to a control subject or population, in one embodiment. Reducing restenosis may comprise reducing the severity of restenosis in a subject, in one embodiment. In important embodiments, the subject is human.

In one embodiment, restenosis occurs following a medical intervention, and non-invasive electrical nerve stimulation is performed after the medical intervention. Non-invasive electrical nerve stimulation is performed 6-24 hours, 1-7 days, or weeks or months after the medical intervention.

In one embodiment, the medical intervention is an intravascular stent placement into a narrowing within the body of the subject. In one embodiment, the intravascular stent placement is an arterial stent placement. In one embodiment, the intravascular stent placement is a venous stent placement. In one embodiment, the intravascular stent placement is a bare-metal stent placement. In one embodiment, the intravascular stent placement is a drug-eluting stent placement.

In one embodiment, the medical intervention is angioplasty.

In one embodiment, the medical intervention is a non-vascular stent placement. In one embodiment, the medical intervention is a esophageal stent placement, a tracheal stent placement, a urethral stent placement, or a bile duct stent placement.

Non-invasive electrical nerve stimulation may be performed one or more times on these and other subjects described herein. Each non-invasive electrical nerve stimulation may be a single cycle or it may be 2, 3, 4, 5 or more cycles of a nerve stimulation period followed by a rest period. In some embodiments, the nerve stimulation period may be up to 1, 2, 3, 4, 5, 6, 7, 8, 9, 10, 15, 20, 25 or 30 minutes in duration. In some embodiments, the rest period may be about 1, 2, 3, 4, 5, or more minutes.

Non-invasive electrical nerve stimulation may be carried out at any body location, including an upper limb (e.g., an arm) or a lower limb (e.g., a leg). Such locations may be local or remote to the site of the ischemic event or the restenosis. Identical or different locations may be used if multiple non-invasive electrical nerve stimulations are performed.

Various of the methods described above may also comprise administering to the subject a chemical or biological agent useful in the treatment of ischemic and/or reperfusion injury whether in the heart or in another organ or tissue. The subjects may be those having or that have had an ischemic event or those having or likely to have restenosis. In some embodiments, the agent is an angiotensin-converting enzyme (ACE) inhibitor. Examples of ACE inhibitors suitable to the invention include but are not limited to captopril, enalapril, ramipril, lisinopril, quinapril, fosinopril, benazepril, and moexipril. In some embodiments, the agent is an angiotensin II receptor blocker. Examples include but are not limited to candesartan, irbesartin, losartin, telmisartin, and valsartan. In some embodiments, the method agent is an anti-platelet agent. Examples include aspirin and clopidogrel. In some embodiments, the agent is a statin. In various embodiments, the subject may be administered two or more agents, including but not limited to two or more of the specific aforementioned agents.

Aspects of the invention provide methods for enhancing physical performance, including but not limited to competitive athletic performance using non-invasive electrical nerve stimulation with or without repetitive exercise. The invention provides in one aspect a method for enhancing physical performance comprising performing non-invasive electrical nerve stimulation on a subject prior to physical activity. The physical activity may be maximal physical activity, or it may be submaximal physical activity.

In some embodiments, the non-invasive electrical nerve stimulation may be performed within 24 hours, within 12 hours, within 6 hours, within 2 hours, or within 20 minutes of the physical activity, without limitation. As used in these aspects of the invention, the term “within” refers to “prior to” the physical activity.

In these and other aspects of the invention, the non-invasive electrical nerve stimulation may be performed one or more time prior to the physical activity. For example, the non-invasive electrical nerve stimulation may be performed at least once daily, or at least once every two days, three days, every four days, every five days, every six days, or every week.

In some embodiments, non-invasive electrical nerve stimulation comprises 1, 2, 3, 4 or 5 cycles a nerve stimulation period followed by a rest period. In some embodiments, the nerve stimulation period may be up to 1, 2, 3, 4, 5, 6, 7, 8, 9, 10, 15, 20, 25 or 30 minutes in duration. In some embodiments, the rest period may be about 1, 2, 3, 4, 5, or more minutes.

In some embodiments, non-invasive electrical nerve stimulation is performed on an upper limb and/or on a lower limb. In some embodiments of these and all other aspects of the invention, non-invasive electrical nerve stimulation is performed at two or more locations on the body, whether in a simultaneous, consecutive, overlapping, or alternating manner.

In some embodiments, the subject is human. In other embodiments, the subject is a non-human including but not limited to a horse or a dog.

In some embodiments, the subject is a healthy subject. In some embodiments, the subject may have cardiovascular disease. In some embodiments, the subject is not experiencing angina and/or has not been diagnosed with angina including refractory angina. In some embodiments, the subject is not experiencing pain such as back pain. In some embodiments, the subject is a competitive athlete.

In some embodiments, the method causes an improvement in physical activity in the range of about 1-20%, 1-10%, or 1-5%. In one embodiment, the method causes about a 1%, 2%, 3%, 4%, or 5% improvement in physical activity. In other embodiments, the method causes about a 0.5%, about a 0.6%, about a 0.7%, about a 0.8%, about a 0.9%, about a 1.0%, about a 1.1%, about a 1.2%, about a 1.3%, about a 1.4%, or about a 1.5% improvement in physical activity.

In another aspect, the invention provides a method for enhancing physical performance comprising performing non-invasive electrical nerve stimulation on a subject having a cardiovascular condition prior to a physical activity in order to enhance performance of the physical activity. In some embodiments, the subject does not have angina and has not been diagnosed with angina. In some embodiments, the subject has angina but is not experiencing pain at the time non-invasive electrical nerve stimulation is performed. The non-invasive electrical nerve stimulation may be performed on a limb such as an arm or a leg.

In another aspect, the invention provides a non-invasive electrical nerve stimulation device comprised in a garment (e.g., between layers of the garment). In one embodiment, garment is athletic apparel. In one embodiment, the system comprises a strap, harness or belt. In one embodiment, the device operates via a remote (or wireless) controller.

In another aspect, the invention provides for use of a non-invasive electrical nerve stimulation device, comprising using the device to perform non-invasive electrical nerve stimulation on a healthy subject prior to a maximal physical activity by the subject.

In another aspect, the invention provides for use of a non-invasive electrical nerve stimulation device, comprising using the device to perform non-invasive electrical nerve stimulation on a subject having a cardiovascular condition prior to a physical activity.

The device and the use of the device in performing non-invasive electrical nerve stimulation enhances performance of the physical activity by the subject.

In one embodiment, the device is a manual device. In one embodiment, the device is an automatic device. In one embodiment, the device comprises a strap, harness or belt.

In one embodiment, the device is comprised in a garment. The garment may be a swimsuit, a running suit, or a ski suit, although it is not so limited.

These and other aspects and embodiments of the invention will be discussed in greater detail herein.

### BRIEF DESCRIPTION OF DRAWINGS

The accompanying Figures are not intended to be drawn to scale. In the Figures, each identical or nearly identical component that is illustrated in various Figures is represented by a like numeral. For purposes of clarity, not every component may be labeled in every Figure.

Various embodiments of the invention will now be described, by way of example, with reference to the accompanying figures, in which:

FIG. 1 is bar graph showing the effect of non-invasive electrical nerve stimulation (dermatomal stimulation), direct femoral nerve stimulation (FN stimulation), deliberate mechanical remote ischemic conditioning (rIPC), and electroacupuncture (EA) on infarct size relative to a sham control.

FIG. 2 is a bar graph showing the effect of non-invasive electrical nerve stimulation (dermatomal stimulation), direct femoral nerve stimulation (FN stimulation), deliberate mechanical remote ischemic conditioning (rIPC), and electroacupuncture (EA) on left ventricular peak systolic and end-diastolic pressure (LVEDP) relative to a sham control.

FIG. 3 is a bar graph showing the effect of non-invasive electrical nerve stimulation (dermatomal stimulation), direct femoral nerve stimulation (FN stimulation), deliberate mechanical remote ischemic conditioning (rIPC), and electroacupuncture (EA) on left ventricular peak diastolic pressure as a percentage of pre-ischemia level (LVDP) relative to a sham control.

FIG. 4 is a bar graph showing the effect of non-invasive electrical nerve stimulation (dermatomal stimulation), direct femoral nerve stimulation (FN stimulation), deliberate mechanical remote ischemic conditioning (rIPC), and electroacupuncture (EA) on the maximal rates of rise in LV pressure (dP/dtₘₐₓ) relative to a sham control.

FIG. 5 is a bar graph showing the effect of non-invasive electrical nerve stimulation (dermatomal stimulation), direct femoral nerve stimulation (FN stimulation), deliberate mechanical remote ischemic conditioning (rIPC), and electroacupuncture (EA) on the maximal rates of fall in LV pressure (dP/dtₘᵢₙ) relative to a sham control.

### DETAILED DESCRIPTION OF INVENTION

The invention relates, in part, to the use of non-invasive electrical nerve stimulation to reduce or prevent ischemic/reperfusion injury in a subject. The invention is based, in part, on the unexpected and surprising finding that non-invasive electrical nerve stimulation is as effective as deliberate mechanical ischemic conditioning in protecting a tissue or organ from ischemic insult. Deliberate mechanical ischemic conditioning, which includes remote ischemic conditioning, has been shown to reduce ischemic and/or reperfusion injury associated with cardiac surgery, vascular surgery and myocardial infarction. The Examples demonstrate that the cardioprotective effects of both deliberate mechanical ischemic conditioning and non-invasive electrical nerve stimulation are mediated through one or more factors in the blood, and that such effects can be transferred to a subject in need of such cardioprotection. That non-invasive peripheral nerve stimulation could provide cardioprotective effects as good as those achieved with deliberate mechanical ischemic conditioning was not heretofore recognized or appreciated in the art.

Ischemic and/or reperfusion injury, as used herein, refers to injury sustained in a subject's body due to ischemia and/or reperfusion associated with an ischemic event. The injury may be in any region, including any organ, of the body, including heart, kidney, liver, pancreas, lung, brain, intestine, spleen, and eyes. The subjects to be treated according to certain aspects of the invention are those that have experienced or are experiencing an ischemic event.

Ischemic and/or reperfusion injury, and thus reductions in ischemic and/or reperfusion injury, may be assessed anatomically and/or functionally, and the nature of such assessments will depend upon the region of the body or organ being treated or protected. As an example, an anatomical assessment of ischemic and/or reperfusion injury may be achieved through imaging and measuring observable tissue injury. As another example, a functional assessment of the ischemic and/or reperfusion injury may be achieved by measuring function of the affected tissue or organ. With respect to the heart, ischemic and/or reperfusion injury may be assessed for example by infarct size or it may be assessed through one or more hemodynamic parameters including for example ejection fraction. Imaging modalities such as computed tomography (CT), magnetic resonance (MR), arteriography, positron emission tomography (PET), and ultrasound including echocardiography can be used to assess a subject. Reducing ischemic and/or reperfusion injury, in some instances, provides long term benefits to a subject. As an example, reducing ischemic and/or reperfusion injury in the heart can ultimately lead to a reduction in the incidence of heart failure, or it can lead to a delay in the onset of heart failure, or it can reduce the severity of heart failure that develops. Accordingly, the invention provides methods for reducing ischemic and/or reperfusion injury that may be manifest in the short term or in the long term.

The invention contemplates protecting tissues and/or organs from ischemic and/or reperfusion injury or reducing the extent of such injury. It will be understood that the ischemic and/or reperfusion injury may exist in a variety of tissues and/or organs. Thus, while various aspects of the invention may be exemplified in the context of myocardial ischemia, the methods provided herein are broadly applicable to other types of tissue or organ ischemia as well.

The invention relates generally to the use of non-invasive electrical nerve stimulation in a variety of subjects including but not limited to those that are experiencing or those that have experienced an ischemic event in a tissue and/or organ of the body. Ischemic events include but are not limited to cardiac ischemic events, cerebral ischemic events, renal ischemic events, pulmonary ischemic events, hepatic ischemic events, pancreatic ischemic events, ocular ischemic events, retinal ischemic events, intestinal ischemic events, and the like. Ischemic events also include acute ischemic conditions such as myocardial infarctions and strokes including transient ischemic stroke and hemorrhagic stroke, as well as chronic ischemic conditions. Ischemic events also include ischemia associated with or resulting from a surgery. The ischemia may occur during the surgery or it may occur after the surgery. Any surgery, regardless of location on the body, is associated with an increased risk of myocardial infarction and stroke post-surgery. This is particularly true in elderly subjects. This may be referred to as “consequential ischemia.” The surgery may be elective or emergency surgery, including but not limited to cardiovascular surgery including vascular surgery, cardiac surgery, stent placements such as intravascular stent placements, angioplasty such as balloon angioplasty, coronary artery bypass graft, heart valve surgery, heart transplantation, surgery for congenital heart disease, as well as lung surgery, liver surgery, kidney surgery, pancreas surgery, colon surgery, bowel surgery, including organ transplant such as but not limited to lung transplant, liver transplant, kidney transplant, and pancreas transplant.

The invention contemplates that short and/or long term benefits can be derived from the use of non-invasive electrical nerve stimulation. For example, with respect to the heart, the methods of the invention provide short term benefits (e.g., the reduction of an infarct size) as well as long term benefits (e.g., the reduction in the likelihood and/or severity of heart failure, or delaying or preventing the onset of heart failure). These and other aspects of the invention will be described in greater detail herein.

As used herein, the term “treat” means to have a positive or therapeutic benefit on a subject that has experienced or is experiencing ischemic and/or reperfusion injury to a tissue and/or an organ. Typically this will involve a reduction in the injury which can be assessed in the short term and/or in the long term. An example of a short term assessment is infarct size resulting from an ischemic event (e.g., myocardial infarct size following a myocardial infarction). Another example of a short term assessment is hemodynamic function (examples of which are provided in the Examples section) following an ischemic event such as a myocardial infarction. In some instances, it is contemplated that the non-invasive electrical nerve stimulation may affect one or more but potentially not all assessments positively. For example, it is possible that infarct size may not be reduced while hemodynamic function may be improved as a result of non-invasive electrical nerve stimulation. The invention further contemplates that non-invasive electrical nerve stimulation may also reduce the likelihood, onset time, and/or severity of chronic injury resulting from the ischemic event and manifest in the long term. An example is congestive heart dysfunction/failure after a myocardial infarction. Positive or therapeutically beneficial effects, in some instances, may be measured by comparing the subject to a population that has not been subjected to the methods of the invention. As an example, the subject and the “untreated” population can be compared in terms of incidence of heart dysfunction/failure, time of onset of heart dysfunction/failure, and severity of heart dysfunction/failure.

### Non-Invasive Electrical Nerve Stimulation

As used herein, non-invasive electrical nerve stimulation may be a single cycle of nerve stimulation followed by a rest period during which no current is applied to the subject, or it may be repeated cycles of nerve stimulation followed by a rest period. The repeated cycles may comprise 2, 3, 4, 5 or more cycles of nerve stimulation followed by a rest period. For clarity, two cycles of non-invasive electrical nerve stimulation would consist of a nerve stimulation period, a rest period, a nerve stimulation period, and a rest period. The invention contemplates that, in some embodiments, a single nerve stimulation period may be sufficient to achieve the desired therapeutic, prophylactic or performance endpoints.

The nerve stimulation period and the rest period may each range from 30 seconds to several minutes or hours. Either or both periods may be up to or about 30 seconds, or 1, 2, 3, 4, 5, 6, 7, 8, 9, 10, 15, 20, 25, 30, 35, 40, 45, 50, 55 or 60 minutes in duration, or longer. The two periods may or may not be of the same duration. An exemplary non-invasive electric nerve stimulation comprises 4 or 5 cycles of 5 minutes of nerve stimulation followed by 5 minutes of rest. Another exemplary non-invasive electrical nerve stimulation comprises 4 or 5 cycles of 4 minutes of nerve stimulation followed by 4 minutes of rest.

The non-invasive electrical nerve stimulation device may be operated under any number of pulse amplitude (or intensity), pulse width, and pulse frequency settings. As an example, the pulse amplitude may range from 1 to 200 mA, including typically from 1 to 100 mA, from 1 to 90 mA, from 1-80 mA, from 1-70 mA, from 1-60 mA, from 1-50 mA, from 1-40 mA, from 1-30 mA, from 1-20 mA, from 1-15 mA, from 1-10 mA, from 1-9 mA, from 1-8 mA, from 1-7 mA, from 1-6 mA, from 1-5 mA, from 1-4 mA, from 1-3 mA, or from 1-2 mA. The pulse frequency may range from 1 to 300 Hz, including typically from 1 to 150 Hz, from 1-140 Hz, from 1-130 Hz, from 1-120 Hz, from 1-110 Hz, from 1-100 Hz, from 1-90 Hz, from 1-80 Hz, from 1-70 Hz, from 1-60 Hz, from 1-50 Hz, from 1-40 Hz, from 1-30 Hz, from 1-20 Hz, from 1-10 Hz, from 1-9 Hz, from 1-8 Hz, from 1-7 Hz, from 1-6 Hz, from 1-5 Hz, from 1-4 Hz, from 1-3 Hz, or from 1-2 Hz. The pulse width may range up to 1 to 1600 microseconds, including typically from 1 to 800 microseconds, from 1-700 milliseconds, from 1-600 milliseconds, from 1-500 milliseconds, from 1-400 milliseconds, from 1-300 milliseconds, from 1-200 milliseconds, from 1-100 milliseconds, and from 1-50 milliseconds. The device may also operate at a voltage typically up to 80 V, including typically up to 40 V, up to 30 V, up to 20 V, up to 10 V, and up to 5 V. The Examples show exemplary settings in which the pulse amplitude is 2-3 mA, the pulse frequency is 3.1 Hz, and the pulse width is 500 microseconds.

Non-invasive electrical nerve stimulation may be performed at any site on the body that is amenable to the non-invasive procedure. It may be performed on any outer surface of the body, including but not limited to arms, legs, feet, hands, torso, chest, back, and the like. It may be performed at a remote site (i.e., a site that is distal to the area of the body experiencing or likely to experience the ischemic and/or reperfusion injury). In other words, the placement of the electrodes may be distal to the region of the body being treated. As an example, the electrodes may be placed on the legs in order to reduce injury in the heart. Typically at least two electrodes are placed within proximity of each other in order to allow current to flow therebetween. Additional paired electrodes may be used at the same or different surface region of the body at the same or different time.

Repeated non-invasive electrical nerve stimulations may be performed at a single, identical site or at multiple, different sites on the body. As an example, a first stimulation may be performed on the right upper arm, followed by a second stimulation performed on the left upper arm. In some embodiments, the non-invasive electrical nerve stimulation is not performed on the chest. Repeated non-invasive electrical nerve stimulations may alternate between two sites or they may cycle through more than two sites. In some instances, non-invasive electrical nerve stimulation may be performed on a subject at two different sites at overlapping times including simultaneously. The use of more than one location may be determined a priori or it may be random. When multiple locations are used simultaneously, two or more devices are typically used.

### Timing of Non-Invasive Electrical Nerve Stimulation Relative to an Ischemic Event

According to some aspects of the invention, non-invasive electrical nerve stimulation may be performed during and after an ischemic event, or after an ischemic event. In preferred embodiments, the non-invasive electrical nerve stimulation is performed repeatedly on the subject, and even more preferably it is performed repeatedly following the ischemic event. The invention contemplates that repeated non-invasive electrical nerve stimulation following an ischemic event provides unexpected benefits to the subject. The subject may or may not have also undergone non-invasive electrical nerve stimulation prior to the ischemic event. The following describes the timing and frequency of non-invasive electrical nerve stimulation in the context of a myocardial infarction. It is intended and should be understood that this description applies to other ischemic events and should be so construed.

When non-invasive electrical nerve stimulation is performed on the subject during, for example, a myocardial infarction, it may be performed during the ischemia that is associated with a myocardial infarction (i.e., the ischemic phase or ischemic period), or during the reperfusion associated with a myocardial infarction (i.e., the reperfusion phase or reperfusion period), or during both phases to the same or to varying degrees.

When the non-invasive electrical nerve stimulation is performed on the subject after, for example, a myocardial infarction (regardless of whether it was also performed during the myocardial infarction), and particularly when it is performed repeatedly, at least one stimulation may be performed within 30 minutes, within 1 hour, within 2 hours, within 3 hours, within 4 hours, within 5 hours, within 6 hours, within 8 hours, within 10 hours, within 12 hours, within 18 hours, or within 24 hours of the end of the ischemic phase of the myocardial infarction. In some embodiments, at least one non-invasive electrical nerve stimulation is performed later than 3 hours after the myocardial infarction including later than 6 hours after the myocardial infarction. In still other embodiments, the non-invasive electrical nerve stimulation may be performed within 36 hours, 48 hours, or 60 hours of the myocardial infarction. The time between the myocardial infarction and the first post-myocardial infarction non-invasive electrical nerve stimulation may be 1, 2, 3, 4, 5, or 6 days, or longer. The totality of non-invasive electrical nerve stimulation may be performed, and thus continued, over any time period including without limitation for up to 1 month, 2, 3, 4, 5, 6, 7, 8, 9, 10, 11, or 12 months, or longer following an MI. In some instances, the regimens occur over years including up to 2, 3, 4, 5, or more years. In still other instances, the regimens continue over the remaining lifespan of the subject.

In some embodiments, the non-invasive electrical nerve stimulation is performed during the reperfusion phase of a myocardial infarction, and then once or several times every day, or every two days, or every three days thereafter, or on prescribed days thereafter. This may be continued to a week, a month, several months, a year, or longer.

It is to be understood that all of the foregoing teachings regarding timing relative to a myocardial infarction apply equally to any other ischemic event and that the invention embraces such other methods.

If non-invasive electrical nerve stimulation is performed during and after an ischemic event such as myocardial infarction, the stimulation that occurs during the event may be referred to as a “first” stimulation and stimulation that occurs after the event may be referred to as subsequent stimulation. However, it is possible that the subject previously experienced non-invasive electrical nerve stimulation prior to the event and the recitation of “first” is not meant to exclude such subjects.

Non-invasive electrical nerve stimulation that occurs after, for example, a myocardial infarction may be performed on a daily basis, every other day (i.e., every two days), every three days, every four days, every five days, every six days, every week, or at longer intervals in time. In some instances non-invasive electrical nerve stimulation may be performed at least once a day, at least once every two days, at least once every three days, at least once every four days, at least once every five days, at least once every six days, at least once every seven days following a myocardial infarction. As used herein, “at least once” as in for example “at least once every three days” means that in a three day period at least one non-invasive electrical nerve stimulation is performed. Similarly, “at least daily” means that every day one or more non-invasive electrical nerve stimulations is performed.

Whether performed on a single day or on multiple days, the non-invasive electrical nerve stimulation may be performed once a day, or more than once a day, including twice a day, 3 times a day, or more.

It will be clear that the time in between non-invasive electrical nerve stimulations may be uniform (or identical) or it may differ. In some instances, such timing will be known ahead of time. The invention also contemplates the performance of multiple randomly spaced non-invasive electrical nerve stimulations.

### Myocardial Infarction

The invention contemplates the use of non-invasive electrical nerve stimulation on subjects that have had one or more myocardial infarctions in the past (i.e., subjects with a history of myocardial infarction) and on subjects who have never had a myocardial infarction prior to being treated according to the methods of the invention. These subjects may be treated according to the invention at the time of the myocardial infarction or shortly thereafter (e.g., within 6-12 hours of the myocardial infarction).

Those of ordinary skill in the art, including but not limited to medical practitioners and medical emergency personnel, will be familiar with the characteristics of an MI. Symptoms of MI, particularly in men, include sudden chest pain (often times radiating to the left arm or left side of neck), shortness of breath, nausea, vomiting, palpitations, sweating, and anxiety. Symptoms in women differ somewhat from those in men, and typically include shortness of breath, weakness, indigestion, and fatigue. Whether in the presence or absence of such symptoms, MI may be detected using, for example, electrocardiograms, blood marker tests (e.g., creatine-kinase, troponin T or I), and heart imaging such as chest X-rays. Guidelines for diagnosing an MI include the WHO criteria (i.e., history of ischemic type chest pain lasting for more than 20 minutes, changes in serial ECG tracings, and rise/fall of serum cardiac markers such as creatine kinase MB and troponin) in which the presence of two and three such criteria indicate probable and definite MI, respectively.

Myocardial infarction symptoms can be contrasted with those of angina pectoris (i.e., angina) in a number of ways. For example, chest pain associated with myocardial infarction is typically spontaneous in onset, is not relieved by rest, lasts longer, and is ultimately associated with observable injury to the heart. In contrast, chest pain associated with angina is typically brought on by physical exertion such as exercise, it is relieved by rest, it is of short duration (e.g., minutes), and it is not associated with permanent injury to the heart.

The invention contemplates the use of non-invasive electrical nerve stimulation during and/or after a myocardial infarction in order to reduce ischemic and/or reperfusion injury. A reduction in ischemic and/or reperfusion injury may be manifest as a reduction in the infarct size or volume following a myocardial infarction. The infarct size may be compared to infarct sizes from the other comparable individuals or to infarct sizes from a population, including a population of comparable individuals that have not been treated according to the invention.

The subjects may also be monitored for their levels of serum biomarkers such as but not limited to troponin, creatine kinase, serum potassium, serum sodium, and serum chloride.

Although not intending to limit the invention to any particular mechanism of action, it is contemplated that, when used in the context of a myocardial infarction, non-invasive electrical nerve stimulation may prevent or restrict the degree of left ventricular remodeling that would otherwise occur in the absence of such treatment. Non-invasive electrical nerve stimulation, and particularly repeated non-invasive electrical nerve stimulation, may attenuate inflammatory responses, reduce oxidative stress, and/or modulate hypertrophic and fibrotic signals associated with myocardial infarction.

It is also contemplated that therapeutic and long term benefits, such as a reduction in the incidence and/or severity of heart failure, may be had regardless of whether there is any observable reduction in infarct size.

Heart failure is generally defined as an impairment in the ability of the heart to pump blood through the body or to prevent blood from backing up into the lungs. Heart failure is often times referred to as congestive heart failure and is associated with systolic or diastolic heart dysfunction. It typically develops over time and may be triggered or exacerbated by another condition that causes heart tissue damage (e.g., an MI) or that causes the heart tissue to work more (or harder) than normal. Heart failure, as used herein, includes but is not limited to the complete cessation of pumping by the heart.

Accordingly, and as will be understood by those of ordinary skill in the art, heart failure indicates heart dysfunction and the invention contemplates reducing the risk, delaying the onset, preventing and/or treating heart dysfunction in the presence or absence of heart failure. The discussion of heart failure herein is therefore intended to capture heart dysfunction also, unless stated otherwise.

The invention provides, in some instances, methods for reducing the risk of heart dysfunction/failure in subjects who have had or are having an MI. The method is intended to reduce the development and/or severity of heart dysfunction/failure as a result of the MI. Development and severity of heart dysfunction/failure can be measured by monitoring and measuring symptoms or other characteristics associated with heart dysfunction/failure. These are discussed below. The methods may lead to the prevention of all or some such symptoms, the delayed onset of all or some such symptoms, and/or the reduction in the severity of all or some such symptoms. A reduction in the risk of heart dysfunction/failure may be determined by monitoring the symptoms or other characteristics associated with heart dysfunction/failure in the treated subject and comparing the number, onset, and severity of such symptoms or characteristics in that subject with historical population data for heart dysfunction/failure. For example, it is known that subjects that survive MI are more likely to develop heart dysfunction/failure than the average population. The methods of the invention aim to reduce this likelihood or risk of heart dysfunction/failure development.

Symptoms of heart dysfunction/failure include shortness of breath (dyspnea), swelling in the feet and legs (edema) typically as a result of abnormal fluid retention, fluid in the lungs, persistent coughing or wheezing, low exercise tolerance, general fatigue even in the absence of exercise, increased heart rate (or palpitations), loss of appetite, memory loss (or confusion), and nausea. One and typically more than one of these symptoms will be manifest in subjects having heart dysfunction/failure. The methods of the invention aim to prevent the development, delay the onset, and/or reduce the severity of one or more of these symptoms.

Heart dysfunction/failure can be diagnosed based on presentation of one and typically more than one of the foregoing symptoms. Heart dysfunction/failure can also be diagnosed or a suspected diagnosis of heart dysfunction/failure can be confirmed with tests such as an electrocardiogram (ECG or EKG), an echocardiogram (“cardiac echo”), or cardiac catheterization. Echocardiograms, for example, are able to measure the volume or fraction of blood that is ejected from the left ventricle with each beat. This is referred to as the ejection fraction. In normal subjects, about 60% of the blood in the left ventricle is ejected. Subjects may present with mildly depressed ejection fractions (e.g., 40-45%), moderately depressed ejection fractions (e.g., 30-40%), or severely depressed ejection fractions (e.g., 10-25%). Thus, in some aspects of the invention, the methods aim to maintain the ejection fraction, particularly if the subject presents with normal or mildly depressed ejection fractions. In some aspects, the methods of the invention aim to delay the onset of a depressed ejection fraction, regardless of the initial ejection fraction presentation. Stress tests may also be used to diagnose heart dysfunction/failure, and they may be combined with one or more of the imaging tests discussed above. For example, a stress test may be combined with an echocardiogram in order to monitor and measure heart dysfunction/failure before, during and/or following exercise periods. Those of ordinary skill in the art, including medical practitioners and more particularly cardiologists, will be familiar these tests and their use in diagnosing heart dysfunction/failure.

### Cardiovascular Surgery

Some aspects of the invention comprise the use of non-invasive electrical nerve stimulation to reduce ischemic and/or reperfusion injury resulting from cardiovascular surgery. The cardiovascular surgery may be performed on the heart and/or on the vasculature. Examples of cardiovascular surgery include but are not limited to heart transplantation, coronary artery by-pass surgery, cardiac valve surgery, surgery for congenital heart disease, carotid artery procedure, vascular grafting, vascular surgery including peripheral vascular surgery, and vascular replacement. Other minimally invasive procedures that are known to induce or likely to induce vessel damage are also considered ischemic events in the context of the invention, and these include stent placement and balloon angioplasty (or percutaneous transluminal coronary angioplasty (PTCA)). The vessel may be a blood vessel such as an artery or a vein.

The surgery or non-surgical procedure may be elective (and thus typically scheduled) or it may done on an emergency basis. The invention contemplates performing non-invasive electrical nerve stimulation during and after, or after the surgery or procedure.

Stent placement or insertion may occur in any vessel of the body including many of the vessels discussed herein, and in any region of the body. Commonly, stent placement occurs intravascularly in an artery or in a vein. Stent placement may also occur in the bile duct, in the esophagus, and in the trachea. Stent placement may be used in any vessel to correct or ameliorate a narrowing of the vessel. The stents may be of any type, including “bare” stents (such as bare-metal stents, used as vascular stents) and drug-eluting stents. Drug-eluting stents, as used herein, refer to stents which are coated with or otherwise comprise one or more therapeutic agents. Bare stents, on the other hand, do not comprise such agents. Bare and drug-eluting stents are known in the art.

### Restenosis

Some aspects of the invention relate to the prevention or treatment of restenosis. Restenosis refers to renarrowing of a vessel or other narrowed biologic structure, and is a common complication following dilatation or stent placement (sometimes referred to as in stent restenosis). It can occur in anywhere from 10-50% of patients. Non-invasive electrical nerve stimulation may be used instead of, or in addition to, a surgical procedure to re-expand a narrowing.

Certain aspects of the invention provide for the use of non-invasive electrical nerve stimulation to reduce the occurrence and severity of restenosis. Restenosis may occur following a medical procedure (or intervention) aimed at opening or widening a blood vessel or biologic tube (including but not restricted to esophagus, biliary tree, bronchus, and the like). Such procedures include but are not limited to stent placements and balloon angioplasty, both of which can cause vessel damage.

Non-invasive electrical nerve stimulation may be performed on a subject that has or that is likely to experience vessel damage that can lead to restenosis. In these subjects, non-invasive electrical nerve stimulation is performed during and after, or after, the occurrence of an event, such as a medical procedure, that is likely to induce vessel damage.

The subjects to be treated according to the invention include those that have undergone a medical intervention that induced or is likely to induce vessel damage. In some instances, these interventions do not themselves produce an ischemic event or environment in the subject.

Medical interventions that are known to induce or are likely to induce vessel damage may be any surgical or non-surgical procedure that results in damage to any vessel in the body. The vessel may be a blood vessel such as an artery or a vein. As used herein, the vessel may be a non-blood vessel (i.e., a vessel that carries a fluid other than, or in addition to, blood) such as the bile duct, the esophagus, the intestine (including large and small intestine), the trachea, the urethra, and the like.

An example of such an intervention is a stent placement (or insertion). Stent placement or insertion may occur in any vessel of the body including many of the vessels discussed herein, and in any region of the body, preferably provided that non-invasive electrical nerve stimulation is performed remotely to the location of the stent. Commonly, stent placement occurs intravascularly in an artery or in a vein. Stent placement may also occur in the bile duct, in the esophagus, and in the trachea. Stent placement may be used in any vessel to correct or ameliorate a narrowing of the vessel.

The stents may be of any type, including “bare” stents (such as bare-metal stents, used as vascular stents) and drug-eluting stents. Drug-eluting stents, as used herein, refer to stents which are coated with or otherwise comprise one or more therapeutic agents. Bare stents, on the other hand, do not comprise such agents. Bare and drug-eluting stents are known in the art.

Another example of a medical intervention is angioplasty (or percutaneous transluminal coronary angioplasty (PTCA)). Restenosis has been reported to occur in 30-50% of subjects who have undergone simple balloon angioplasty.

Non-invasive electrical nerve stimulation may be performed during and after or after the medical intervention. Non-invasive electrical nerve stimulation is typically performed, in whole or in part, after the medical intervention. In such instances, non-invasive electrical nerve stimulation may be performed 6-24 hours (including 6-12 hours), 1-7 days, weeks, or months after the medical intervention.

In some embodiments, non-invasive electrical nerve stimulation is performed on a number of days, including 2, 3, 4, 5, 6, 7, 8, 9, 10, 15, or 30 or more days, or 1, 2, 3, 4, 5, 6 or more months. It is to be understood that in such instances, a subject may undergo non-invasive electrical nerve stimulation daily, every 2, 3, 4, 5, or 6 days, every week, every 2, 3, 4 weeks, every month, every 2, 3, 4, 5, 6 months, for example. Additionally, non-invasive electrical nerve stimulation may be performed in a non-regular, or random, manner.

Certain aspects of the invention therefore intend to reduce the occurrence (or incidence) of restenosis in a subject, and/or to reduce the severity or degree of the restenosis, and/or to reduce or ameliorate the symptoms associated with restenosis.

A reduced occurrence of restenosis can be determined by comparing the treated subject to another subject, or more preferably a population of subjects, that has not received non-invasive electrical nerve stimulation but is otherwise medically comparable to the treated subject. The average time of restenosis in this control group is compared to that of the treated subject, and a delayed onset of restenosis in the treated subject relative to the control is indicative of a reduced occurrence.

A reduction in the severity or degree of restenosis may be measured directly or indirectly. For example, the severity or degree of restenosis may be measured directly through, for example, measurement of a vessel diameter. Indirect measurements may include functional measurements. The nature of the functional measurement will depend upon the nature and normal function of the damaged vessel. An example of a functional measurement is flow rate and flow quality through the vessel. These measurements are preferably made when the restenosis is likely to occur, based on historical data from comparable but untreated subjects.

Analysis of symptoms relating to restenosis will also depend on the nature of the vessel(s) that may restenose. If restenosis may occur in the vasculature, then symptoms include any cardiovascular symptoms relating to blood flow impairment, including but not limited to cardiac and cerebral symptoms. These may include unusual fatigue, shortness of breath, and chest pressure.

Biological markers may also be measured as an indicator of restenosis. An example of a biological marker is troponin, which is elevated in the presence of restenosis.

Various tests are available to detect restenosis including imaging tests (e.g., CT, radionuclide imaging, angiography, Doppler ultrasound, MRA, etc.), and functional tests such as an exercise stress test.

### Additional Therapies

In some aspects of the invention, non-invasive electrical nerve stimulation may be used in combination with other therapies or procedures. In some instances, the therapies or procedures are used to reduce the risk or severity of heart damage and/or heart dysfunction/failure. These therapies include without limitation anti-platelet drug therapy including fibrinolytic agents, anti-coagulation agents, and platelet function inhibitors, beta blocker therapy, ACE inhibitor therapy, statin therapy, aldosterone antagonist therapy (e.g., eplerenone), and omega-3-fatty acids therapy. Depending upon the embodiment, one or more of these agents may be administered before, at the time of, or after MI, whether or not overlapping with the non-invasive electrical nerve stimulations. These and other suitable therapies are discussed in greater detail below.

Fibrinolytic agents are agents that lyse a thrombus (e.g., a blood clot), usually through the dissolution of fibrin by enzymatic action. Examples include but are not limited to ancrod, anistreplase, bisobrin lactate, brinolase, Hageman factor (i.e. factor XII) fragments, molsidomine, plasminogen activators such as streptokinase, tissue plasminogen activators (TPA) and urokinase, and plasmin and plasminogen.

Anti-coagulant agents are agents that inhibit the coagulation pathway by impacting negatively upon the production, deposition, cleavage and/or activation of factors essential in the formation of a blood clot. Anti-coagulant agents include but are not limited to vitamin K antagonists such as coumarin and coumarin derivatives (e.g., warfarin sodium); glycosoaminoglycans such as heparins both in unfractionated form and in low molecular weight form; ardeparin sodium, bivalirudin, bromindione, coumarin dalteparin sodium, desirudin, dicumarol, lyapolate sodium, nafamostat mesylate, phenprocoumon, sulfatide, tinzaparin sodium, inhibitors of factor Xa, factor TFPI, factor VIIa, factor IXc, factor Va, factor VIIIa as well as inhibitors of other coagulation factors.

Inhibitors of platelet function are agents that impair the ability of mature platelets to perform their normal physiological roles (i.e., their normal function). Examples include but are not limited to acadesine, anagrelide, anipamil, argatroban, aspirin, clopidogrel, cyclooxygenase inhibitors such as nonsteroidal anti-inflammatory drugs and the synthetic compound FR-122047, danaparoid sodium, dazoxiben hydrochloride, diadenosine 5′,5′″-P1,P4-tetraphosphate (Ap4A) analogs, difibrotide, dilazep dihydrochloride, 1,2- and 1,3-glyceryl dinitrate, dipyridamole, dopamine and 3-methoxytyramine, efegatran sulfate, enoxaparin sodium, glucagon, glycoprotein IIb/IIIa antagonists such as Ro-43-8857 and L-700,462, ifetroban, ifetroban sodium, iloprost, isocarbacyclin methyl ester, isosorbide-5-mononitrate, itazigrel, ketanserin and BM-13.177, lamifiban, lifarizine, molsidomine, nifedipine, oxagrelate, PGE, platelet activating factor antagonists such as lexipafant, prostacyclin (PGI₂), pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612 and ZK-36374, 2,4,5,7-tetrathiaoctane, 2,4,5,7-tetrathiaoctane 2,2-dioxide, 2,4,5-trithiahexane, theophyllin pentoxifyllin, thromboxane and thromboxane synthetase inhibitors such as picotamide and sulotroban, ticlopidine, tirofiban, trapidil and ticlopidine, trifenagrel, trilinolein, 3-substituted 5,6-bis(4-methoxyphenyl)-1,2,4-triazines, and antibodies to glycoprotein IIb/IIIa as well as those disclosed in U.S. Pat. No. 5,440,020, and anti-serotonin drugs, Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine.

In some instances, the therapies or procedures are used to reduce inflammation associated with certain conditions such as restenosis. Anti-inflammatory agents include without limitation Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium. One preferred anti-inflammatory agent is aspirin.

Lipid reducing agents include gemfibrozil, cholystyramine, colestipol, nicotinic acid, probucol lovastatin, and statins such as fluvastatin, simvastatin, atorvastatin, pravastatin, and cirivastatin.

Direct thrombin inhibitors include hirudin, hirugen, hirulog, agatroban, PPACK, thrombin aptamers.

Glycoprotein IIb/IIIa receptor inhibitors are both antibodies and non-antibodies, and include but are not limited to ReoPro (abcixamab), lamifiban, tirofiban.

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, aminone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and the like and/or pharmaceutically acceptable salts thereof.

Beta-adrenergic receptor blocking agents (also known as beta blockers) are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

A number of selective “COX-2 inhibitors” are known in the art. These include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors”; U.S. Pat. No. 5,677,318 Diphenyl-1, 2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-dihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include but are not limited to peptidic compounds (e.g., saralasin, [(San¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4, 5, 6, 7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(−1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl] 1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G.D.Searle and Company).

ACE inhibitors include amino acids and derivatives thereof, peptides, including di- and tri-peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart dysfunction/failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di- and tri-peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

HMG-CoA reductase inhibitors include, but are not limited to, statins such as simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402, the disclosures of which patents are incorporated herein by reference.

It is to be understood that the invention contemplates the use of one or more of any of the foregoing agents in combination with non-invasive electrical nerve stimulation.

### Performance Enhancement

Other aspects of the invention are directed to the use of non-invasive electrical nerve stimulation to enhance physical performance in subjects. These aspects of the invention are directed towards subjects who desire an improvement or enhancement of their level of physical activity or performance.

In some instances, the invention is directed even more specifically to athletes, including competitive athletes. Such subjects are under a tremendous pressure to improve performance times and/or other judged end points without the use of prohibited performance enhancing drugs. The invention contemplates that non-invasive electrical nerve stimulation would satisfy this need as it does not involve administration of any banned substance and instead simply takes advantage of inherent processes that operate in the body naturally. These subjects may be swimmers, short distance or long distance track runners, marathon runners, skiers, cyclists, and the like.

These aspects of the invention are not limited solely to athletic subjects and instead can be applied to any subject that will perform a physical activity and in whom an improved performance is desired. The subjects may have average and possibly even below average athletic abilities yet would still be suited for the methods described herein. In some instances, the subjects are healthy. In some embodiments, the subjects may have poor heart function, heart failure, or other circulatory disturbances that might limit exercise performance. The subjects may or may not have angina including angina pectoris.

Such subjects will preferably be humans, although non-human subjects are also contemplated. Such non-human subjects include but again are not limited to any animal used in strenuous competition (e.g., racing) such as horses and dogs.

In one aspect, the invention provides a method for enhancing physical performance comprising performing non-invasive electrical nerve stimulation on a subject prior to a physical activity. In another aspect, the invention provides a method for enhancing physical performance comprising performing non-invasive electrical nerve stimulation on a healthy subject prior to a maximal physical activity. The methods of the invention can be used as a long-term training regimen. Similarly devices and systems that perform non-invasive electrical nerve stimulation according to the invention may be used as training aids.

The non-invasive electrical nerve stimulation may be performed as described above under “Non-Invasive Electrical Nerve Stimulation.” Briefly, it may be performed as a single cycle of a nerve stimulation period followed by a rest period, or it may be multiple such cycles including but not limited to 2, 3, 4 or 5 cycles. Each period may range from about 30 seconds to several minutes, including for example up to 30 seconds, up to 1, 2, 3, 4, 5, 6, 7, 8, 9, 10 or more minutes, in duration. The nerve stimulation and rest periods may or may not be of the same duration. An exemplary non-invasive electric nerve stimulation may comprise a single cycle of 5 minutes of nerve stimulation followed by a rest period of any duration. Another exemplary non-invasive electrical nerve stimulation may comprise 4 or 5 cycles of up to 4 or 5 minutes of nerve stimulation followed by up to 4 or 5 minutes of rest.

Again, as taught herein, the non-invasive electrical nerve stimulation may be carried out on any location of the body. It may be carried out on any surface of the body, including any skin surface. For ease and convenience, it may be carried out on a limb such as an arm or leg, but it is not so limited. In some embodiments, it is not carried out on the chest.

In some embodiments, non-invasive electrical nerve stimulation is performed prior to and typically not during the physical activity. It may be performed within 48 hours, within 24 hours, within 12 hours, within 6 hours, within 4 hours, within 2 hours, within 1 hour, within 30 minutes, within 20 minutes, within 10 minutes, or within 5 minutes prior to the physical activity, or just immediately prior to the physical activity. It may be performed one or more times, in one day, or per day (daily), or on prescribed days over the course of days, weeks, or months. If two or more non-invasive electrical nerve stimulations are performed, they need not be identical to each other with respect to timing, number of cycles, and the like.

In some aspects of the invention, the method is intended to improve the performance of a maximal physical activity. As used herein, the term maximal physical activity means an activity in which the subject exerts itself maximally. Exertion levels may be measured in a number of ways known in the art including but not limited to heart rate range, the “talk test”, and the Borg rating of perceived exertion (RPE). The degree of activity that yields maximal exertion may vary between certain subjects based on age and physical condition. Nevertheless, methods exist in the art to determine for each subject the level of activity that corresponds to moderate, vigorous or maximal exertion.

The following is a method for determining the level of activity being performed for a given individual using heart rate. Generally, the person's age is subtracted from the hypothetical maximum heart rate of 220. The resulting number is multiplied by a percentage based upon the level of activity being performed. Moderate intensity activity corresponds to about 50-70% of the “age-adjusted” maximum heart rate. Vigorous intensity activity corresponds to 70-85% of the “age-adjusted” maximum heart rate. Maximal activity corresponds to anything higher than 85% of the age-adjusted maximum heart rate.

If the Borg RPE is used, a score of 19 or 20 corresponds to maximal exertion, a score in the range of 15-18 corresponds to vigorous exertion, and a score in the range of 12-14 corresponds to moderate exertion.

In still other embodiments, particularly those which involve subjects with cardiovascular disease, exercise may be limited. In these and other similar situations, an exercise intensity level of NYHA (New York Heart Association) grade 2-4 is contemplated.

Examples of moderate intensity activity include but are not limited to walking briskly (3 miles per hour or faster), water aerobics, bicycling slower than 10 miles per hour, ballroom dancing, tennis (doubles), and general gardening.

Examples of vigorous intensity activity include but are not limited to race walking, jogging or running (e.g., marathon running or racing), swimming laps, tennis (singles), aerobic dancing, bicycling 10 mile per hour or faster, biathlons, triathlons, or other single or multiple activity competitions (e.g., Iron Man competitions), diving such as deep sea diving, free diving and base diving, jumping rope, heavy gardening (e.g., continuous digging or hoeing), hiking uphill or with a heavy backpack, and the like.

The activity to be benefited according to the invention may be short (e.g., 60 minutes or less, including 5, 10, 20, 30, 40, 50 or more minutes) or it may be long (e.g., more than one hour, including 2, 3, 4, 5, 6 or more hours) in duration.

Physical activity that can also benefit from the methods of the invention includes the activity associated with a rescue operation such as a coast guard rescue operation (e.g., a rescue at sea), activity associated with first-responder activity (e.g., rescuing persons from a burning building), activity associated with hand-to-hand combat military missions, and the like.

Maximal intensity activity could typically be any of the vigorous intensity activities recited herein provided they are performed at the individual subject's maximal ability (i.e., an “all-out” attempt).

It is to be understood that the invention provides methods for improving performance that occurs for any of the foregoing activities since whether a particular activity will require moderate, vigorous or maximum exertion will depend on the individual and their physical ability and condition.

It is also to be understood that the invention contemplates using non-invasive electrical nerve stimulation to improve performance for submaximal activities also. The invention contemplates that subjects less physically fit than competitive athletes will also benefit from non-invasive electrical nerve stimulation, for example, when performing submaximal activity.

The methods for measuring performance enhancement will vary based on the particular activity being performed. For example, if the activity is swimming, then the enhancement may be measured by the time to swim a certain distance (e.g., 50 meters, 100 meters, or more). If the activity is running, then the enhancement may be measured by the time to run a certain distance (e.g., 50 meters, 100 meters, 200 meters, 1 mile, a marathon, etc.). Similarly, if the activity is cycling, speed skating, and the like, then the enhancement may be measured by the time taken to traverse a certain distance. It will be understood that in these examples, the enhancement will be manifested as a decrease in the time taken to perform the activity in question. Other suitable endpoints and readouts will be apparent to those of ordinary skill in the art.

The degree of performance enhancement that can be achieved using the methods provided herein may vary between individuals. The degree of performance enhancement will typically be measured using the difference between the endpoints or readouts achieved following non-invasive electrical nerve stimulation and a sham control. The quotient of that difference and the sham control readout is representative of the improvement achieved. As an example, a 1% enhancement is a decrease of a second for an activity that would take on average 100 seconds to perform in the absence of non-invasive electrical nerve stimulation.

In some instances, the degree of enhancement may be on the order of 0.1%-1%, including 0.5%-1% yet still be statistically significant and more importantly competitive or physiologically significant. In still other instances, the degree of enhancement may be up to 1.5%, up to 2%, up to 2.5%, up to 3%, up to 3.5%, up to 4%, up to 4.5%, up to 5%, up to 10%, up to 20%, up to 30%, up to 40%, up to 50%, or more.

Accordingly, various aspects of the invention provide methods to improve resistance to exercise-induced fatigue in healthy individuals during sports and activities, and in patients limited by cardiac, circulatory or other medical disorders (e.g., patients with heart failure, peripheral vascular disease, lung disease) that may limit blood flow or muscle power.

### Deliberate Mechanical Ischemic Conditioning

The invention further contemplates that non-invasive electrical nerve stimulation may be used with, or in subjects who have undergone, are undergoing, or will undergo, deliberate mechanical ischemic conditioning. As used herein, deliberate mechanical ischemic conditioning refers to a deliberate induced ischemic event followed by a reperfusion event. A deliberate mechanical ischemic conditioning may be performed as a single cycle (i.e., one ischemic event followed by one reperfusion event) or as multiple cycles. Multiple cycles include but are not limited to two, three, four, five or more cycles. Deliberate mechanical ischemic conditioning is typically performed by restricting blood flow in a limb or a peripheral tissue of the subject and then removing the blood flow restriction and allowing blood to reperfuse the limb or tissue. Deliberate mechanical ischemic conditioning is typically non-invasive.

The blood flow restriction typically takes the form of an applied pressure to the limb or tissue that is above systolic pressure (i.e., supra-systolic pressure). It may be about 5, about 10, about 15, about 20, or more mmHg above (or greater than) systolic pressure. Since systolic pressure will differ between subjects, the absolute pressure needed to induce ischemia will vary between subjects. In other embodiments the pressure may be preset at, for example, 200 mmHg. The blood flow restriction may be accomplished using any method or device provided it is capable of inducing transient ischemia and reperfusion, whether manually or automatically. Such devices include without limitation a manually inflatable cuff, a tourniquet system, or an automated device as described below.

The induced ischemic event is transient. That is, it may have a duration of about 1, about 2, about 3, about 4, about 5, or more minutes. Similarly, the reperfusion event may have a duration of about 1, about 2, about 3, about 4, about 5, or more minutes.

If performed using a limb, one or both the upper limbs or lower limbs may be used although in some instances one or both the upper limbs is preferred. In some instances, deliberate mechanical ischemic conditioning is performed on two different sites on the body, in an overlapping or simultaneous manner.

### Devices

Devices for performing non-invasive electrical nerve stimulation are known in the art. An example of such as device is a transcutaneous electrical nerve stimulation system or device. A suitable device minimally comprises two or more electrodes, a frequency potentiometer, a current potentiometer, and a power supply. The frequency potentiometer is used by the operator to adjust the frequency of the electrical signal. The current potentiometer is used by the operator to adjust the current that is output from the system and delivered to the subject. It will be apparent that the operator of the device may also be the end user including the subject being treated. The invention contemplates devices that are used non-invasively. In preferred embodiments, the devices are not implantable devices and not implanted in the subject. Additionally, such devices are typically not connected to other implantable or implanted devices such as pace makers and the like.

The device may be particularly suited for use in an athletic environment. As an example, it may be configured in a manner to be secured to a body location such as an arm or a leg. As an example, it may be associated with an elasticized housing or it may be elasticized itself. Alternatively or additionally, it may be attached to or incorporated into clothes including socks, shoes, running apparel (e.g., running suits, running jackets, or running pants), swimming apparel (e.g., swim suits, including fastskin suits, etc.), and the like, in whole or in part. In the case of a full body suit, the device may be in the arm(s) and/or in the leg(s) part of the suit. The suit may be so configured as to provide an external cord or outlet to or into which a power cord may be connected or to which other elements of the device may be connected (e.g. the controller, the power source, and the like). Alternatively the suit may be configured such that the device, in whole or in part, can be threaded between two materials or between two layers comprised in the suit. In still other embodiments, a shoulder harness and/or a belt may be associated with the device in order to provide support. The device may comprise a waterproof housing, or it may be otherwise designed to be waterproof, or it may have waterproof elements. The device may comprise a housing that protects it from bodily fluids such as sweat (e.g., it may be “sweat-proof”). In these and other embodiments, the device may comprise a plastic or other water-resistant housing.

In the event the device is used for animals such as racing breeds (e.g., dogs, horses, etc.), it may be suitably configured for such use. As an example, the device may be provided or encased in the covers (e.g., capes) used to keep such animals warm during training or prior to competition.

As used herein, a garment is any form of clothing or apparel. The garment may be clothing or apparel that is worn during physical activity or during a warm-up period prior to physical activity. The device (or system) may be provided on an inner layer or surface of the garment so that it contacts the subject. The device (or system) may be provided between layers of the garment so that it does not contact the subject. When provided in association with a garment, the device (or system) may or may not comprise the electrodes. In some instances, the device will not comprise a power source (e.g., batteries and/or cords) and/or the device will not comprise a controller. The device may be connected to a controller and/or a power source when non-invasive electrical nerve stimulation is to be performed. The incorporation of a device (without a controller and/or a power source) will therefore reduce or limit the mass (or weight) added to the garment, thereby allowing the garment to be worn throughout training and performance time periods. The electrodes may be disposable, in some instances. The garment may be provided together with a portable, self-contained (stand alone) controller and/or a portable, self-containing (stand alone) power source or supply. It will therefore be understood that one or more elements of the device (e.g., the electrodes) may be associated with a garment (and therefore be referred to as integral to or incorporated within the garment), and/or that one or more elements of the device (e.g., the power source or supply) may be physically separate from the garment. The invention contemplates a kit that comprises the garment and any physically separate elements of the device, including the controller and/or the power source or supply.

The invention further contemplates operation of the device through direct connections or wirelessly. Wireless operation may comprise wireless control of the device. As an example, the controller may be physically separate from the electrodes although they may be in wireless contact with each other. This configuration allows the electrodes to be controlled at a distance, and may reduce the overall weight of the device. Wireless controllers include mobile devices including smart phones and other wireless hand-held devices that can be programmed and/or can upload computer applications that will control the operation of the device. The wireless controller may also direct the power source or supply to turn off and/or on. Non-limiting examples of commercially available wireless controllers are an iPhone™, an iPod™, an iPad™ a Blackberry™, and the like. As will be clear, such wireless devices will allow for remote control of the device. In such instances, the device may further comprise an override mechanism that allows the subject wearing the garment (or someone in the vicinity) to override a remote instruction.

The invention further contemplates a hybrid device that is capable of performing non-invasive electrical nerve stimulation and deliberate mechanical ischemic conditioning. Such a hybrid device may comprise at least two electrodes and a cuff, under the control of one or more controllers, and optionally a power source.

Devices for performing deliberate mechanical ischemic conditioning are also known in the art. An example of such as device is a sphygmomanometer (i.e., the instrument typically used to measure a subject's blood pressure). Another example of such as device is a manual type tourniquet. Devices such as those described in published PCT application WO 83/00995, in published US application 20060058717, and in published US application 20080139949 may also be used. This latter system comprises a cuff configured to retract about a limb of a subject, an actuator connected to the cuff that when actuated causes the cuff to contract about the limb of the subject to reduce blood flow there through, and a controller that controls the actuator according to a treatment protocol. The treatment protocol typically includes a plurality of treatment cycles, each of which may comprise a cuff actuation period during which the actuator contracts the cuff about the limb of the subject to a pressure above systolic pressure to occlude blood flow through the limb, an ischemic duration period during which the actuator maintains the cuff contracted about the limb at a set point above systolic pressure to occlude blood flow through the limb, a cuff release period during which the actuator releases the cuff to allow blood flow through the limb, and a reperfusion duration period during which the cuff is maintained about the limb in a relaxed state to allow blood flow through the limb.

### EXAMPLES

This example compares the effect of non-invasive dermatomal electrical nerve stimulation, femoral nerve stimulation, and electroacupuncture on ischemic/reperfusion damage in the heart.

Dermatomal stimulation was performed using a transcutaneous electrical nerve stimulation (TENS) device by applying electrode pads directly onto the medial side of the hindlimb of a rabbit subject. The TENS device was set to deliver a stimulus having a 2.0-3.0 mA pulse intensity, a 3.1 Hz pulse frequency, and a 500 microsecond pulse width. Four cycles of five minutes of stimulation followed by a five minute rest period were performed.

Femoral nerve stimulation as performed using a TENS device by applying an electrode needle directly adjacent to the femoral nerve of a rabbit subject. The TENS device was set to deliver a stimulus having a 0.5-1.0 mA pulse intensity, a 3.1 Hz pulse frequency, and a 500 microsecond pulse width. Four cycles of five minutes stimulation followed by a five minute rest period were performed.

Electroacupuncture was applied bilaterally at the Neiguan point in a rabbit subject.

Disperse-dense waves of alternating frequencies of 2 and 15 Hz were applied at an intensity level of 1-2 mA for 60 minutes. The current intensity was adjusted to maintain a slight twitch of the limb.

Remote ischemic conditioning was also performed on control rabbit subjects. This was performed by four cycles of five minutes of blood flow occlusion in the rabbit hindlimb followed by a five minute rest period.

Sham controls were sedated/anesthetized in identical manner to the other groups, and underwent an identical protocol to those receiving dermatomal stimulation with shaving, fixation of electrodes, etc., but no electrical stimulation was delivered.

Following these various procedures, blood was collected from each of the subjects and a dialysate was prepared from each of the blood samples using a previously described method (Shimizu et al. Clinical Science, 117: 191-200, 2009). To test their effect on heart damage, the hearts of donor rabbits were perfused with the dialysate, following which each of the donors was subjected to thirty minutes of global ischemia followed by 120 minutes of reperfusion. Ischemia and reperfusion induction, heart harvest, and assessment of heart damage by infarct size estimation. were carried out as previously described (see Steensrud et al. Am J Physiology 2010).

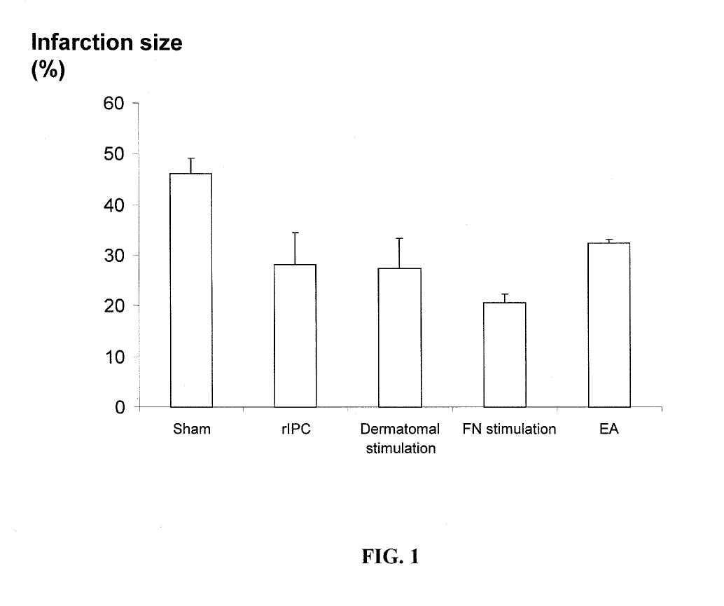

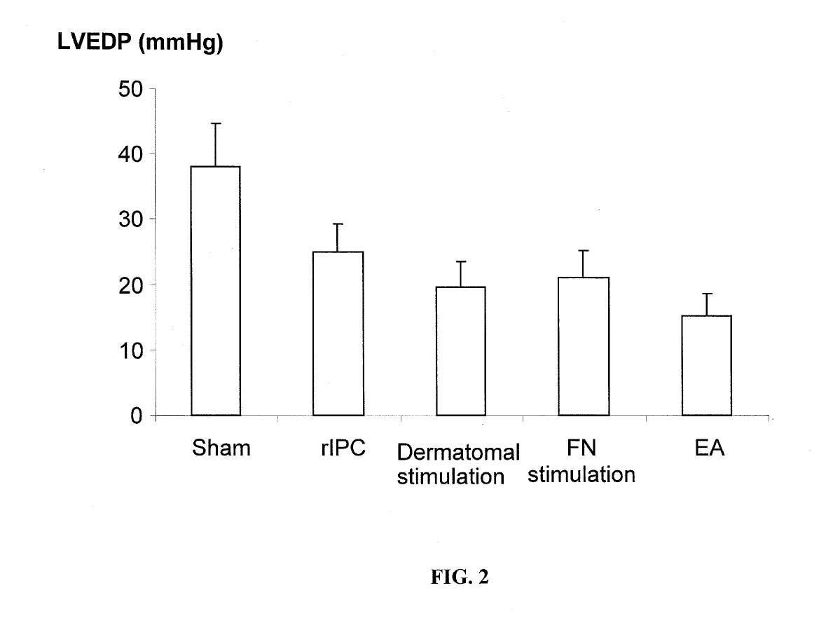

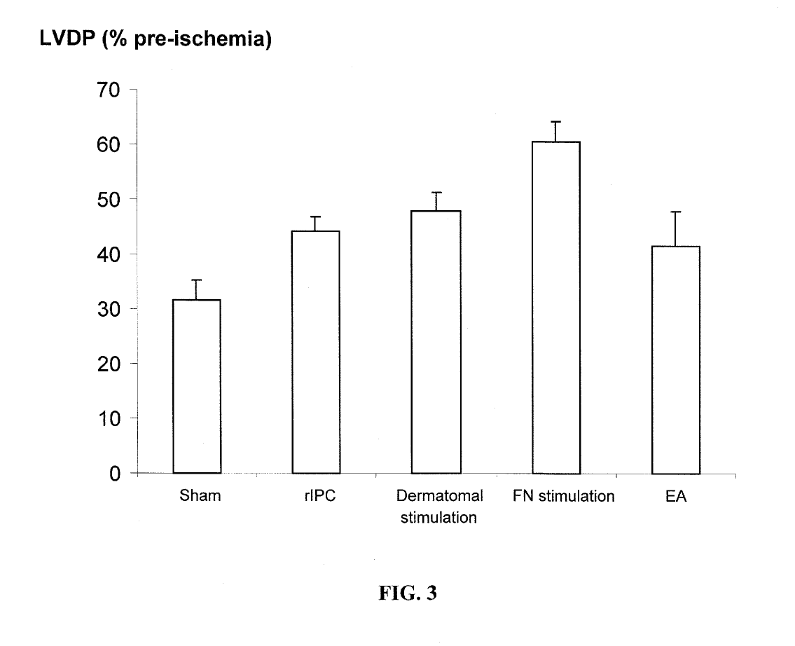

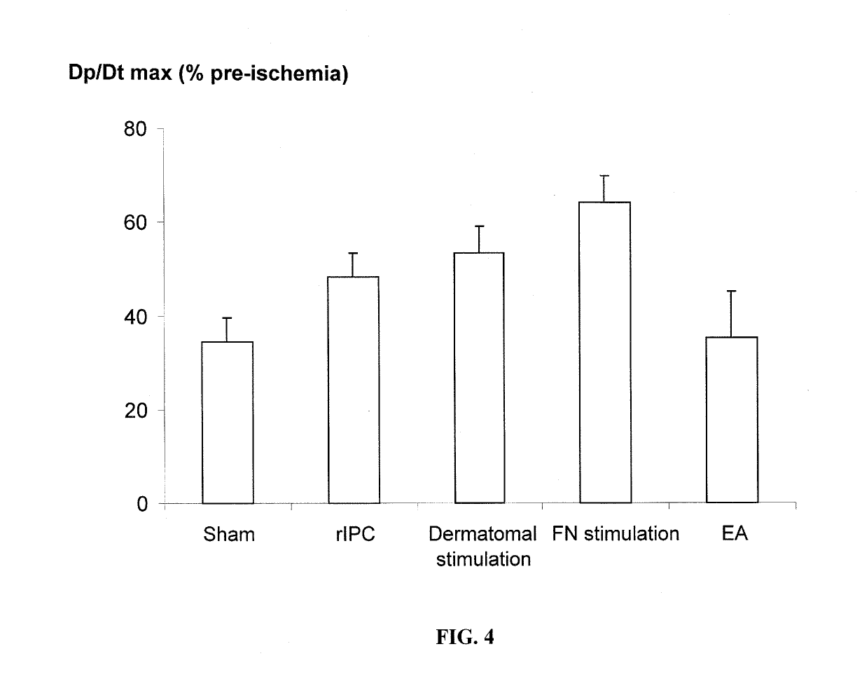

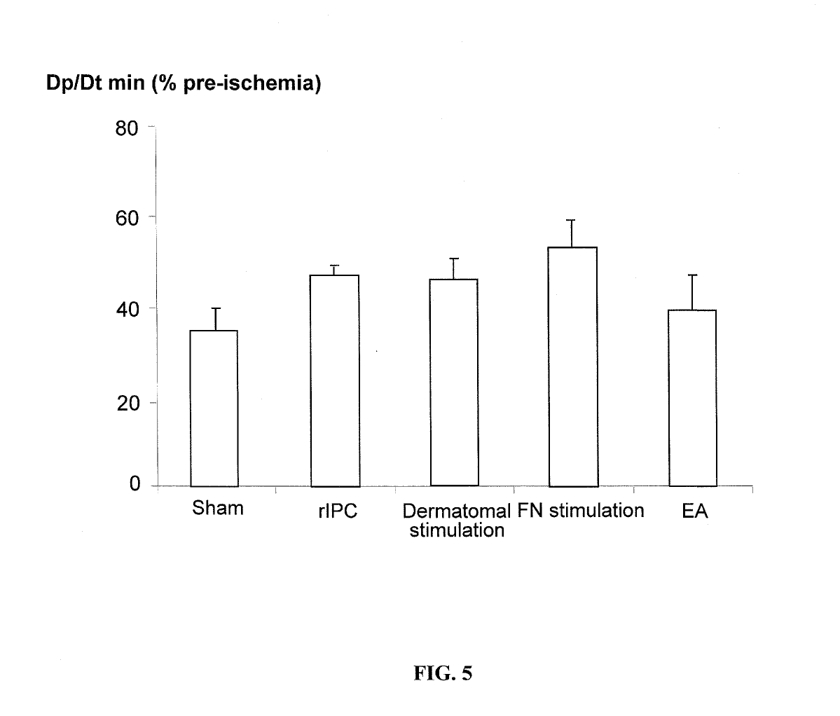

The results are shown in FIGS. 1-5. As shown in the Figures, dialysate from subjects that experienced remote ischemic conditioning (rIPC), dermatomal stimulation, femoral nerve stimulation (FN stimulation), and electroacupuncture (EA) reduced infarct size relative to the sham control. In this and other measurements of heart function, including LVEDP, LVDP, dp/dtₘₐₓ and dp/dtₘᵢₙ), dermatomal stimulation was at least as effective as remote ischemic conditioning in reducing ischemic/reperfusion damage to the heart. This non-invasive dermatomal stimulation was also as effective, for some parameters, as invasive femoral nerve stimulation.

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

What is claimed is:

1. A method comprising
performing non-invasive electrical nerve stimulation on a subject to reduce ischemic and/or reperfusion injury in the subject associated with an ischemic event, wherein the non-invasive electrical nerve stimulation is performed on the subject repeatedly after the ischemic event.

2. The method of claim 1, wherein the subject is having a myocardial infarction.

3. The method of claim 2, wherein non-invasive electrical nerve stimulation is also performed during the myocardial infarction.

4. The method of claim 2, wherein the non-invasive electrical nerve stimulation is performed during the myocardial infarction and then every day, every two days, or every three days after the myocardial infarction.

5. The method of claim 2, wherein the non-invasive electrical nerve stimulation is continued for 1 month after the myocardial infarction.

6.-9. (canceled)

10. The method of claim 1, wherein the subject is having surgery.

11. The method of claim 10, wherein the non-invasive electrical nerve stimulation is also performed during the surgery.

12. The method of claim 10, wherein the non-invasive electrical nerve stimulation is performed during the surgery and then every day, every two days, or every three days after the surgery.

13. The method of claim 10, wherein the non-invasive electrical nerve stimulation is continued for 1 month after the surgery.

14. The method of claim 10, wherein the surgery is cardiovascular surgery.

15. The method of claim 1, wherein the non-invasive electrical nerve stimulation is 1, 2, 3, 4, or 5 cycles of a nerve stimulation period followed by a rest period.

16. The method of claim 15, wherein the nerve stimulation period is up to 1, 2, 3, 4, 5, 6, 7, 8, 9, 10, 15, 20, 25 or 30 minutes in duration.

17. The method of claim 16, wherein the rest period is 1, 2, 3, 4 or 5 minutes in duration.

18. The method of claim 15, wherein the non-invasive electrical nerve stimulation is 1 cycle of a 5 minute nerve stimulation period followed by a rest period.

19. The method of claim 15, wherein the non-invasive electrical nerve stimulation is 4 or 5 cycles of a 5 minute nerve stimulation period followed by a 5 minute rest period.

20. The method of claim 1, wherein the subject is human.

21. (canceled)

22. The method of claim 1, wherein the non-invasive electrical nerve stimulation is performed transcutaneously.

23.-26. (canceled)

27. A method for reducing restenosis in subject comprising
performing non-invasive electrical nerve stimulation on a subject having or likely to experience restenosis following a medical intervention, wherein the non-invasive electrical nerve stimulation is performed repeatedly after the medical intervention.

28.-48. (canceled)

49. A method for enhancing physical performance comprising
performing a remote ischemic preconditioning regimen on a subject having a cardiovascular condition prior to a physical activity in order to enhance performance of the physical activity.

50. A method for enhancing physical performance comprising
performing non-invasive electrical nerve stimulation on a healthy subject prior to a maximal physical activity.

51.-64. (canceled)
